(19) United States
(12) Patent Application Publication
(10) Pub. No.: US 2019/0350743 A1
(43) Pub. Date: Nov. 21, 2019

(54) Reinforced support tape

(71) Applicants: Conner Fialkowski, Edmond, OK (US); Zachariah Blew, Edmond, OK (US); Easton Hendrickson, Edmond, OK (US)

(72) Inventors: Conner Fialkowski, Edmond, OK (US); Zachariah Blew, Edmond, OK (US); Easton Hendrickson, Edmond, OK (US)

(21) Appl. No.: 15/932,894

(22) Filed: May 16, 2018

(51) Int. Cl. A61F 5/40; A61F 5/01; A61F 13/02

(52) U.S. Cl. CPC A61F 5/40; A61F 5/0104; A61F 13/066; A61F 13/0273; A61F 13/025

(57) ABSTRACT

An adhesive reinforced support tape is provided to support muscles and joints and reduce exerting stress to the covered area, providing a support tape having a rear surface with an adhesive coating for application to the skin surface and a linear pocket for the insertion of a stiffening member into the pocket after the adhesive surface has been applied to the desired area, providing support and limitation of movement to reduce injury during lifting or heavy exertion, ideally suited for patient care where lifting of patients is required.

### CROSS REFERENCE TO RELATED APPLICATIONS

None.

### I. BACKGROUND OF THE INVENTION

### 1. Field of Invention

An adhesive reinforced support tape is provided to support muscles and joints and reduce exerting stress to the covered area, providing a support tape having a rear surface with an adhesive coating for application to the skin surface and a linear pocket for the insertion of a stiffening member into the pocket after the adhesive surface has been applied to the desired area, providing support and limitation of movement to reduce injury during lifting or heavy exertion, ideally suited for patient care where lifting of patients is required.

### 2. Description of Prior Art

A preliminary review of prior art patents was conducted by the applicant which reveal prior art patents in a similar field or having similar use. However, the prior art inventions do not disclose the same or similar elements as the present reinforced support tape, nor do they present the material components in a manner contemplated or anticipated in the prior art.

Body adhesive tape is used for athletes and others who are exposed to injury from strain or lifting. Several of these provide a tape substrate with an adhesive surface to secure the tape to the section of the body where protection is desired. In U.S. Pat. No. 9,308,115 to Quinn, kinesiology tape is provided which has a removable backing to protect the adhesive material prior to use. In U.S. Pat. No. D743,566 to Arbesman, an adhesive brace with a locating window is shown in the drawings. Release liner gridlines are shown on an upper surface of a strip of kinesiology tape in U.S. Pat. No. D737,986, also to Arbesman. Arbesman also demonstrates two more variations for pre-cut kinesiology tape in U.S. Pat. Nos. 9,833,351 and 8,742,196. None of these define a pocket for receiving a stave or support member within a pocket in the tape.

It is intended that the tape be used as a primary support member instead of vests or braces commonly used at the present time. It may also be used in conjunction with the use of a vest or brace as a secondary support member, providing an additional tactical sensory means that the vest or brace would provide to actively alert the medical professional of the extent of the strain prior to injury.

### II. SUMMARY OF THE INVENTION

Each year, numerous medical professionals injure their backs and other body parts trying to assist those patients in need of medical or therapeutic care that have physical restrictions, requiring lifting, carrying or movement by the medical professional under stress. The purpose of the present adhesive reinforced support tape is intended to address the injury potential by a reduction of stress on the applied body part by the inclusion of a support member within a pocket on the support tape which is applied to the area of the body under stress or at risk. The present support tape conforms to the body while also keeping some degree of resistance to bending as a reminder to avoid overexertion or over-extension of the taped area while also being more protected from injury.

### III. DESCRIPTION OF THE DRAWINGS

The following drawings are submitted with this utility patent application.

FIG. 1 is a view of the support tape worn in the center of a person's back.

FIG. 2 is a front view of the support tape.

FIG. 3 is a sectional view of the support tape indicating the support stave located within the tape pocket.

FIG. 4 is a rear view of the support tape showing the adhesive covering prior to the application of the support tape.

### IV. DESCRIPTION OF THE PREFERRED EMBODIMENT

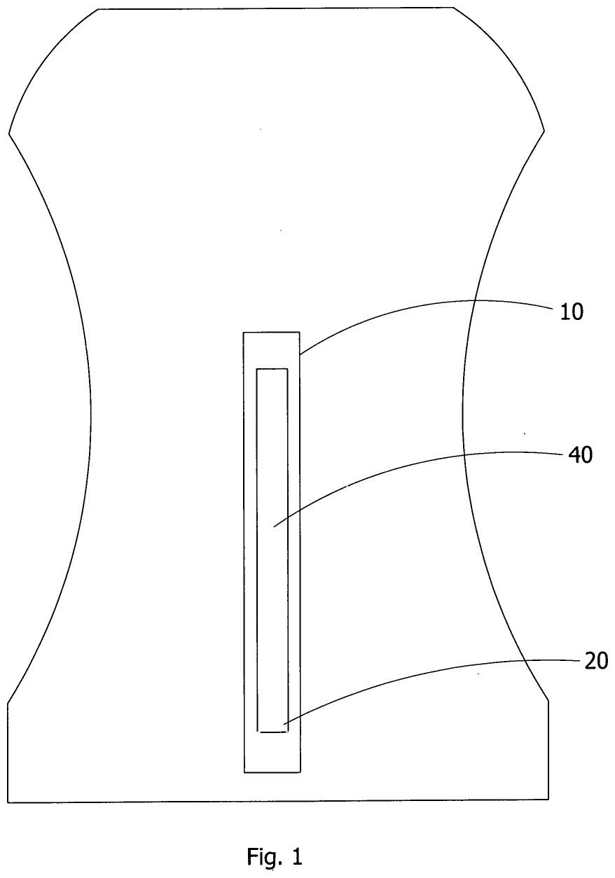

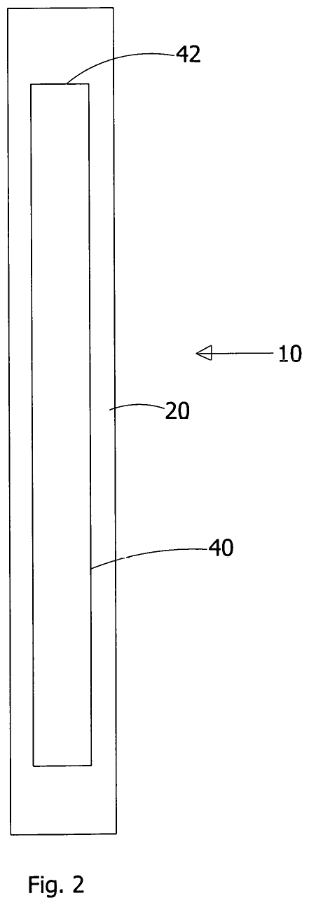

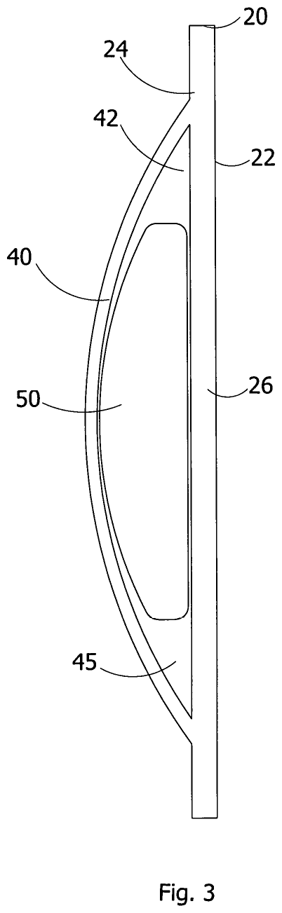

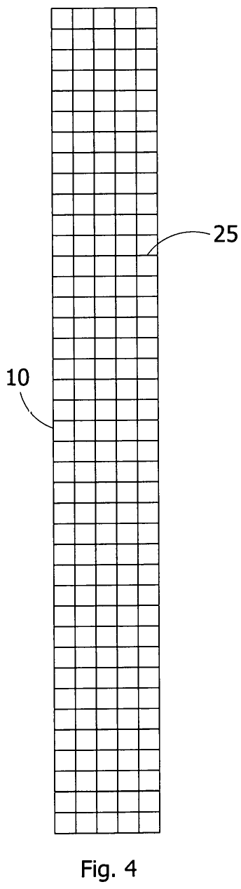

A support tape 10 for application to the skin overlying a joint or muscle, shown in FIGS. 1-4, provides limited support to the joint or muscle but also a reminder to the person wearing the support tape of the potential for overexertion.

The support tape comprises a rear panel 20, made from a fabric or polymeric material having a rear surface 22 and a front surface 24. The rear surface 22 is provided with an adhesive material 26 which adheres to skin. Over the adhesive material 26, prior to use, is an adhesive covering 25 to maintain the adhesive material 26 in a sticky state until the adhesive material 26 is applied to the skin, at which time the adhesive covering 26 is removed. The front surface 24 secures a stave panel 40 applying three sides of the stave panel 40 to the front surface 24 leaving one side 42 of the stave panel 40 open to form a stave pocket 45 between the stave panel 40 and the front surface 24 for installation of a stiffening support stave 50 conforming to the shape of the stave pocket 45. The rear panel 20 would can be applied prior to insertion to the installation of the support stave 50 or subsequent to the installation of the support stave 50.

The support tape 10 and rear panel 20 may be provided in a diversity of shapes and sizes, dependant upon the location of the support tape 10 and the joint over which the support tape 10 is to be applied. The support tape 10 and support stave 50 may be rectangular as shown in the drawing figures or may be presented as curved circular or other shape, provided that there is an open side 42 in the stave pocket 45 and the support stave 50 has the capacity to be inserted into the stave pocket 45 before or after application. Preferable material selection for the components would be a material similar to an adhesive bandage for the support tape 10, including plastic, cloth, a polymer, gauze, or paper, and a stiff material similar to a collar stave for the support stave 50, being a hard but flexible plastic, dense cardboard or even a lightweight metal product.

The limited nature of support is defined by the support tape 10 and support stave 50 not actually preventing an injury to the covered joint or body part, but to warn the user of the stress being applied to the affected joint by tugging and pulling on the skin covering the joint. The support tape 10 and support stave 50 also provide for some degree of limitation of movement of the affected area by the application of tension over the covered area. For excessive movement or stress, there would be an enhanced tugging and pulling on the skin and for light movement, a subtle tugging and pulling on the skin, both the enhanced and subtle tugging being sensed by the person wearing the support tape 10. The support tape 10 provides a tactile alert to the person wearing the support tape 10 that excessive stress on the affected area should be avoided, limited or scaled back.

While the support tape 10 and inserted support staves 50 have been particularly shown and described with reference to a preferred embodiment thereof, it will be understood by those skilled in the art that changes in form and detail may be made therein without departing from the spirit and scope of the invention.

What is claimed is:

1. A support tape for application to the skin overlying a joint or muscle provides limited support to the joint or muscle and a tactile reminder to the person wearing said support tape of a potential for overexertion and over extension, said support tape comprising:
a rear panel made from a fabric or polymeric material defining a rear surface and a front surface;
said rear surface having an adhesive material with an adhesive covering prior to use to maintain said adhesive material in a sticky state until application of said adhesive material at which time said adhesive covering is removed; and
said front surface securing a stave panel applying three sides of said stave panel to said front surface leaving one side of said stave panel open forming a stave pocket between said stave panel and said front surface for installation of a stiffening support stave, said support stave conforming to said slave pocket, wherein said rear panel is applied prior to insertion of said support stave in some applications or subsequent to the installation of the support stave in others.

2. The support tape of claim 1 further comprising:
said support tape and rear panel are provided in a diversity of shapes and sizes, dependant upon the location of said support tape and the joint over which said support tape is applied;
said rear panel and said support tape are made from a material selected from a list including plastic, cloth, a polymer, gauze, or paper; and
said stiffening material of said support stave is a hard but flexible material selected from a list including plastic, dense cardboard or a lightweight metal product.

3. The support tape of claim 1 further comprising:
any other element of feature as shown or indicted within the specification and drawings herein.
